(19) United States
(12) Patent Application Publication
(10) Pub. No.: US 2007/0134349 A1
(43) Pub. Date: Jun. 14, 2007

(54) Methods of treating hematological malignancies

(75) Inventors: Donald P. McDonnell, Chapel Hill, NC (US); Michelle S. Jansen, Durham, NC (US); Huey-Jing Huang, San Diego, CA (US)

Correspondence Address: NIXON & VANDERHYE, PC, 901 NORTH GLEBE ROAD, 11TH FLOOR, ARLINGTON, VA, 22203, US

(73) Assignee: DUKE UNIVERSITY, Durham, NC (US)

(21) Appl. No.: 11/523,091

(22) Filed: Sep. 19, 2006

Related U.S. Application Data
(60) Provisional application No. 60/717,737, filed on Sep. 19, 2005.

(51) Int. Cl. A61K 33/36; A61K 31/203; A61K 31/20; A61K 31/19

(52) U.S. Cl. 424/623; 514/557; 514/559; 514/575

(57) ABSTRACT

The present invention relates, in general, to methods of treating malignancies and, in particular, to methods of treating hematological malignancies and to compositions suitable for use in such methods.

This application claims priority from U.S. Provisional Application 60/717,737, filed Sep. 19, 2005, the entire content of which is incorporated herein by reference.

### TECHNICAL FIELD

The present invention relates, in general, to methods of treating malignancies and, in particular, to methods of treating hematological malignancies and to compositions suitable for use in such methods.

### BACKGROUND

Acute promyelocytic leukemia (APL) accounts for about 10-15% of acute myeloid leukemia (AML). The leukemic cells from most APL patients have a t(15;17) translocation that fuses the PML (promyelocytic leukemia) gene on chromosome 15 to the retinoic acid receptor α (RARα) gene on chromosome 17, resulting in the formation of a PML-RARα chimeric protein (de The et al, Cell 66:675-684 (1991), Kakizuka et al, Cell 66:663-667 (1991)). PML-RARα is thought to function as a dominant negative inhibitor of wild type RARα and PML and in doing so blocks myeloid cell differentiation (Melnick and Licht, Blood 93:3167-3215 (1999)). Two current standard therapies for APL, all-trans-Retinoic acid (ATRA) and arsenic trioxide (ATO), target the PML-RARα fusion protein for degradation (Chen et al, Blood 89:3345-3353 (1997), Raelson et al, Blood 88:2826-2832 (1996)). However, in most patients, resistance to these agents generally arises as a result of an increase in ATRA catabolism or mutations within the PML-RARα protein (Gallagher, Leukemia 16:1940-1958 (2002)). Of late, a great deal of effort has been focused on the identification of agents that relieve the differentiation block imposed on APL cells by the ATRA-resistant PML-RARα mutant proteins. It has become clear in recent years that the aberrant recruitment of histone deacetylases (HDACs) plays a critical role in leukemogenesis (Kramer et al, Trends Endocrinol. Metab. 12:294-300 (2001)). Indeed, HDAC inhibitors (HDACIs) have been shown to induce differentiation and apoptosis in a number of leukemic cell lines (Kramer et al, Trends Endocrinol. Metab. 12:294-300 (2001)). Furthermore, HDACIs was shown to induce remission in an ATRA-resistant, ATO-resistant APL animal model (He et al, J. Clin. Invest. 108:1321-1330 (2001)), suggesting that HDAC inhibitors may provide new therapeutic strategies to overcome resistance to ATRA and ATO in APL. Although it was initially believed that these agents functioned primarily at the level of PML-RARα, it is their ability to induce differentiation and apoptosis in cells that do not express this fusion protein that has led to the suggestion that their mechanism of action is much more complex. Recently, it was shown that HDACIs can induce apoptosis in AML cells in a TRAIL (TNF-Related Apoptosis-Inducing Ligand)-dependent manner but that HDACI-induced differentiation in these cells is TRAIL-independent (Nebbioso et al, Nat. Med. 11:77-84 (2005)). Thus, it appears that HDACIs have at least two distinct pharmacological activities that contribute to their therapeutic efficacy in leukemias. Not surprisingly, there is a high level of interest in determining the molecular mechanism of action of HDACIs in leukemia with a view to improving the therapeutic utility of existing drugs and directing the development of compounds with improved therapeutic efficacy.

Recent evidence suggests that the mitogen-activated kinase (MAPK) pathway might play an important role in the function of HDACIs. MAPK signaling is common to pathways that regulate the proliferation and differentiation in diverse cell types including hematopoietic cells (Platanias, Blood 101:4667-4679 (2003)). In myeloid cells, MEK/ERK signaling has been shown to be important for differentiation (Miranda et al, Leukemia 16:683-692 (2002)). Constitutive activation of ERK is observed in primary AML blasts and leukemia cell lines, and downregulation of ERK activity induces apoptosis of these cells (Lunghi et al, Leukemia 17:1783-1793 (2003), Morgan et al, Blood 97:1823-1834 (2001), Towatari et al, Leukemia 11:479-484 (1997)). HDACIs such as butyric acid (BA), valproic acid, and trichostatin A (TSA) have been reported to activate MAPK (Yang et al, J. Biol. Chem. 276:25742-25752 (2001), Yuan et al, J. Biol. Chem. 276:31674-31683 (2001), Zhong et al, Oncogene 22:5291-5297 (2003)). However, BA can also downregulate MAPK signaling in some systems (Davido et al, Eur. J. Cancer Prev. 10:313-321 (2001), Jung et al, Cancer Lett. 225:199-206 (2005), Witt et al, Blood 95:2391-2396 (2000)). It is also not clear if MAPK activity is linked to the HDAC inhibitor activity of HDACIs. Recently, methoxyacetic acid (MAA) has been shown to inhibit HDAC activity and activate MAPK in HeLa cells (Jansen et al, Proc. Natl. Acad. Sci. USA 101:7199-7204 (2004)), but its effects on leukemia cell differentiation and apoptosis has not been examined.

The present invention results from studies designed to investigate the role of the MAPK pathway and HDAC inhibition in APL cell differentiation and apoptosis induced by MAA and other HDACIs. These studies have revealed that HDACIs induce differentiation and apoptosis through two distinct mechanisms; at low concentrations these agents induce differentiation in an ERK-dependent manner, whereas at higher concentrations they promote apoptosis and inhibit differentiation by quantitatively inhibiting ERK phosphorylation. These previously unappreciated complexities in HDACI action have important clinical implications.

### SUMMARY OF THE INVENTION

In general, the present invention relates to methods of treating malignancies. More specifically, the invention relates to methods of treating hematological malignancies and to compositions suitable for use in such methods.

Objects and advantages of the present invention will be clear from the description that follows.

### BRIEF DESCRIPTION OF THE DRAWINGS

FIGS. 1A-1D. MAA induces differentiation of NB4 cells independent of PML-RARα signaling pathway. NB4 cells were treated with ATRA or MAA for 3 days before analysis for expression of cell surface markers CD11c (FIG. 1A) and CD11b (FIG. 1B) by flow cytometry. (FIG. 1C) NB4 cells were transfected with the indicated reporters along with pCMV-βgal plasmid and treated with or without ATRA or MAA. Cells were harvested 19 h after transfection/treatment and luciferase and β-galactosidase activities were measured. Normalized luciferase values are represented as a ratio of luciferase activity to β-galactosidase activity. *, p<0.05 compared with untreated control (Ctrl) as analyzed by one-way ANOVA with Dunnett's post-test. (FIG. 1D) NB4 cells were treated with ATRA, MAA, and ATO at the indicated concentrations for 3 days and whole cell extracts were analyzed for expression of PML-RARα and RARα by western blotting using anti-RARα antibody. Expression of α-tubulin was also analyzed as a loading control. NS indicates nonspecific band.

FIG. 2. Inhibition of MEK/ERK activity reduces phosphorylation of C/EBPβ induced by HDACIs. Cells were treated with ATRA, MAA, BA with or without U0126 (5 μM) for 17 h before analysis for expression of phospho-C/EBPβ(pC/EBPβ), C/EBPβ, phospho-ERK1/2 (pERK1/2), and ERK1/2 by Western blot analysis.

FIGS. 3A-3C. MAA and BA induce a dose-dependent dual effect on NB4 differentiation and apoptosis. NB4 cells were treated with ATRA (RA) at 1 μM, MAA and BA at the indicated concentrations for 24 h before analysis for expression of (FIG. 3A) cell surface marker CD11c and (FIG. 3B) Annexin V binding by flow cytometry. (FIG. 3C) Whole cell extracts were analyzed for expression of phospho-C/EBPβ(pC/EBPβ), C/EBPβ, phospho-ERK1/2 (pERK1/2), ERK1/2, acetyl-H4 (Ac-H4), acetyl-H3 (Ac-H3), phospho-H3 (pH3), Aurora B, and GAPDH.

FIGS. 4A-4B. MEK/ERK activity is required for NB4 cell differentiation and protects these cells from apoptosis. NB4 cells were pretreated with U0126 at the indicated concentrations for 2 h before ATRA, MAA, or BA was added. Cells were treated with ATRA, MAA, or BA for 21 h before analysis for expression of (FIG. 4A) cell surface marker CD11c and (FIG. 4B) Annexin V binding by flow cytometry. Treatments with the same letter were not significantly different as determined with one-way ANOVA with Tukey's post-test (p<0.05).

FIGS. 5A-5C. MAA and BA trigger similar signal transduction events in U-937 cells. U-937 cells were treated with ATRA (RA) at 1 μM, ATO at 1 μM, MAA and BA at the indicated concentrations for 3 days before analysis for expression of (FIG. 5A) cell surface marker CD11c and (FIG. 5B) Annexin V binding by flow cytometry. (FIG. 5C) U-937 cells were treated with MAA or BA at the indicated concentrations for 18 h and whole cell extracts were analyzed for expression of phospho-ERK1/2, ERK1/2, acetyl-H3, phospho-H3, and GAPDH.

FIGS. 6A-6D. MAA induces differentiation in PML-RARα-negative leukemia cells and ATRA-resistant APL cells. (FIG. 6A) HL-60 cells were treated with ATRA, MAA, or BA for 48 h before analysis for expression of cell surface marker CD11c by flow cytometry. (FIG. 6B) Kasumi-1 cells were treated with MAA or BA for 3 days before analysis for expression of cell surface marker CD11c by flow cytometry. NB4 or NB4-R4 cells were treated with ATRA or MAA for 3 days before analysis for expression of cell surface markers CD11c (FIG. 6C) and CD11b (FIG. 6D) by flow cytometry. *, p<0.05; **, p<0.01 compared with untreated control (Ctrl) in each individual cell line as analyzed by one-way ANOVA with Dunnett's post-test.

FIGS. 7A-7D. MAA potentiates the effects of ATRA and ATO on NB4 cell differentiation and apoptosis. NB4 cells were treated with increasing concentrations of ATRA with or without MAA for 24 h before analysis for expression of cell surface markers CD11c (FIG. 7A) and CD11b (FIG. 7B) by flow cytometry. NB4 cells were treated with increasing concentrations of ATO with or without MAA for 3 days before analysis for expression of cell surface marker CD11c (FIG. 7C) and Annexin V binding (FIG. 7D) by flow cytometry. Treatments with the same letter were not significantly different as determined with one-way ANOVA with Tukey's post-test (p<0.05).

FIG. 8. A working model for HDACI action in differentiation and apoptosis of APL cells. HDACIs exhibit a dose-dependent dual effect on differentiation and apoptosis in NB4 cells. When used at lower concentrations at which no significant histone acetylation is observed, HDACIs increase cell differentiation and phosphorylation of C/EBPβ, both of which require ERK activity in cells. At higher concentrations, HDACIs dramatically downregulate ERK activity and induced apoptosis which is correlated with hyperacetylation of histones H3 and H4, as predicted from their HDACI activity. Phosphorylation of H3 is also increased with higher concentrations of HDACIs, which may result from the increased Aurora B expression level.

FIGS. 9A and 9B. Comparison of the differentiating (FIG. 9A) and apoptotic (FIG. 9B) activities of MAA and VPA.

FIGS. 10A and 10B. Differentiating activities of short chain fatty acids.

### DETAILED DESCRIPTION OF THE INVENTION

The present invention relates to a method of treating hematological malignancies, including leukemias, such as APL (AML-M3), AML-M2 with t(8;21) chromosomal translocation PMLRAR-positive and PMLRAR-negative APL (AML-M3) and ATRA-resistant APL. The method comprises administering to a mammal (human or non-human) in need of such therapy a short chain fatty acid that is both a MAPK activator and an HDAC inhibitor in an amount sufficient to effect the therapy. MAPK activity is required for induction of differentiation whereas HDAC inhibitory activity is important for induction of apoptosis (e.g., of leukemia cells). The invention includes methods of treating a hematological malignancy (e.g., leukemia) in a mammal (e.g., a human) who has become refractory to other forms of treatment. The short chain fatty acids of the invention (e.g., MAA) can also be used as a first-line therapy, for example, in combination with ATRA and ATO to lower doses needed to treat APL. For other subtypes of AML, the short chain fatty acids (e.g., MAA) can be used as a first-line therapy either alone or in conjunction with chemotherapy (the standard therapy for AML).

Short chain fatty acids suitable for use in the invention include C₃-C₁₂ fatty acids, preferably C₃-C₁₀, more preferably C₃-C₈, for example, MAA, butyric acid (BA), valproic acid (VPA), propionic acid, 3-methoxypropionic acid and ethoxyacetic acid, or pharmaceutically acceptable salts thereof.

The short chain fatty acids of the invention can be administered alone or in combination with other chemotherapeutic agents suitable for use in treating hematological malignancies. For example, the short chain fatty acid(s) can be used before, during or after the administration of chemotherapeutic agents including but not limited to arsenic compounds, such as arsenic trioxide or melarsoprol or arsenic sulfides (see, for example, U.S. Appln. 20040146583 and U.S. Pat. No. 6,733,792), and ATRA. In a specific embodiment, the short chain fatty acid and the arsenic compound and/or ATRA is administered as a mixture.

Any suitable mode of administration can be used in accordance with the present invention including but not limited to parenteral administration, such as intravenous, subcutaneous, intramuscular and intrathecal administration; oral, and intranasal administration, and inhalation. The mode of administration can vary, for example, with type of malignancy, and the condition of the mammal.

The invention includes pharmaceutical compositions comprising one or more short chain fatty acid and a carrier. The compositions can be, for example, in the form of a sterile aqueous or organic solution or a colloidal suspension. The composition can also be in dosage unit form, for example, as a tablet. The compositions can comprise additional active agents, such as the chemotherapeutic agents noted above.

The short chain fatty acids of the invention can be used in the treatment of a variety of hematological malignancies. In a preferred embodiment, the malignancy is a leukemia. In addition to APL, examples of applicable leukemias include but are not limited to AML and other undifferentiated leukemias, such as myelodysplastic syndrome (MDS). In addition, the short chain fatty acids of the invention can also be expected to be useful in the treatment of leukemias characterized by the presence of terminally differentiated cells. The methods of the instant invention are also applicable to reduce the number of preneoplastic cells in a mammal in which there is an abnormal increase in the number of preneoplastic cells.

The invention also relates to kits suitable for use in practicing the method of the invention. Such kits can comprise in one or more container means therapeutically effective amounts of one or more short chain fatty acid in pharmaceutically acceptable form. The kit can also comprise an additional chemotherapeutic agent in pharmaceutically acceptable form. The kit can further comprise a needle or syringe for injecting the short chain fatty acid.

The optimal therapeutic dose of a short chain fatty acid can vary, for example, with the short chain fatty acid, the patient and the effect sought and can be readily determined by one skilled in the art. A daily dose of the short chain fatty acid can be from about 0.1 to about 150 mg per kg body weight per day (e.g., parenterally or orally). A preferred daily dose can be from about 1 to about 100 mg/kg body weight of short chain fatty acid, more preferably, from about 10 to about 20 mg/kg/day. Again, any suitable route of administration can be employed for providing the mammal with an effective dosage of the short chain fatty acid. For example, oral, transdermal, iontophoretic, parenteral (e.g., subcutaneous, intramuscular, and intrathecal) can be employed. Dosage unit forms include tablets, troches, cachet, dispersions, suspensions, solutions, capsules and patches. (See, for example, Remington's Pharmaceutical Sciences.)

Compounds (e.g., short chain fatty acids) suitable for use in treating leukemias such as APL can be identified by assaying candidate compounds for their the ability to increase the percentage of AML cell models (e.g. NB4 cells or other appropriate cell type described in the Example that follows) that express the myeloid differentiation markers CD11b and CD11c. Such an assessment can be made, for example, using flow cytometry analysis. This ability has been shown to be associated with the effectiveness of HDAC inhibitors in the treatment of APL.

Certain aspects of the invention can be described in greater detail in the non-limiting Example that follows.

### EXAMPLE

Experimental Details

Materials

ATRA, MAA, BA, ATO, and U0126 were purchased from Sigma (St. Louis, Mo.). Anti-RARα (C-20), anti-C/EBPβ (C-19), anti-phospho-ERK (Tyr-204; E-4), and anti-GAPDH (V-18) antibodies were from Santa Cruz Biotechnology (Santa Cruz, Calif.). Anti-phospho-C/EBPβ (Thr235) and anti-Aurora B antibodies were from Cell Signaling Technology (Beverly, Mass.). Anti-REK1/2 antibody was from Promega (Madison, Wis.). Anti-phospho-Histone H3 (Ser10), anti-acetyl-Histone H3 (Lys9/14), and anti-acetyl-Histone H4 (Lys 5, 8, 12, 16) antibodies were from Upstate Biotechnology (Lake Placid, N.Y.).

Cell Culture and Treatment

NB4 cells were provided by Dr. Ronald Evans (Salk Institute, La Jolla, Calif.). NB4-R4 cells were provided by Dr. Wilson Miller (McGill University, Montreal, Canada). HL-60, U-937, and Kasumi-1 cells were obtained from the American Type Culture Collection (Rockville, Md.). NB4 and NB4-R4 cells were grown in RPMI medium 1640 containing 10% fetal bovine serum. U-937 cells were grown in RPMI medium 1640 containing 10 mM HEPES, 1 mM sodium pyruvate, 4.5 g/L glucose, and 10% fetal bovine serum. HL-60 cells were grown in Iscove's Modified Dulbecco's medium containing 20% fetal bovine serum. Kasumi-1 cells were grown in RPMI medium 1640 containing 10 mM HEPES, 1 mM sodium pyruvate, 4.5 g/L glucose, and 20% fetal bovine serum.

Analysis of Cell Surface Markers and Annexin V Binding

To determine the percentage of cells that express differentiation markers, cells were washed once and resuspended in RPMI medium 1640 containing 3% fetal bovine serum. Phycoerythrin (PE)-conjugated CD11b, 5 Allophycocyanin (APC)-CD11c or control antibodies (BD Pharmingen, San Diego, Calif.) were added into cell suspension. After 30 minutes of incubation in the dark at 4° C., the cells were washed twice and resuspended in RPMI medium 1640 containing 7-amino actinomycin D (AAD) (for dead cell exclusion) and analyzed by FACScan (Becton Dickinson).

To determine the percentage of cells that undergo apoptosis, after incubation with APC-CD11c antibody in the dark at 4° C., cells were washed twice with PBS and incubated at room temperature for 15 min with PE-conjugated Annexin-V (BD Pharmingen) and 7-AAD in binding buffer (10 mM HEPES, pH 7.4, 140 mM NaCI, 2.5 mM CaCl₂) and analyzed by FACScan for CD11c expression and Annexin V binding. Apoptotic cells are those that stained positive for Annexin V and negative for 7-AAD.

Western Blot Analysis

Whole-cell lysates were prepared by washing the cells with PBS and resuspending them in 1 ml of lysis buffer (1× phosphate-buffered saline, 1 mM EDTA, 1.5 mg/ml of iodoacetamide, 100 μM sodium orthovanadate, 0.5% Triton X-100, 20 mM β-glycerolphosphate, 0.2 mM phenylmethylsulfonyl fluoride, and 1× complete protease inhibitor cocktail). After clarification by a 15-min centrifugation in a microcentrifuge at 4° C., the resulting supernatant was collected. 20-50 μg of protein extracts were separated on a 10% SDS-polyacrylamide gel and transferred to a nitrocellulose membrane. The membrane was blocked with 5% nonfat milk followed by incubation with antibodies. The immunocomplex was visualized by chemiluminescence.

Transient Transfection

NB4 cells were grown to a density of 0.6 to 0.9×10⁶ cells/ml, rinsed once with RPMI medium 1640, and 10⁷ cells were resuspended in 0.7 ml of RPMI medium 1640 at room temperature in electroporation cuvettes. 5 μg of TK-Luc or RARE-TK-Luc and 5 μg of pCMV-βgal plasmid were incubated with the cells at room temperature for 5-10 min. Electroporation was performed using a Gene Pulser (BioRad, Hercules, Calif.) apparatus at 300 V and 960 μF capacity. After electroporation, cells were resuspended in 3 ml of media with or without 1 μM ATRA or 5 mM MAA. Cells were harvested 19 h after transfection/treatment for luciferase and β-galactosidase activities.

Statistical Analysis

Statistical calculations were performed using the GraphPad Prism computer program (San Diego, Calif.). One-way ANOVA was used to test whether differences between treatments were significant. If differences were significant (P<0.05), Dunnett's or Tukey's multiple comparison test was then used for post-hoc evaluation of differences between treated and control groups.

Results

HDACI-mediated Differentiation of Acute Promyelocytic Leukemia Cells Occurs Independent of PML-RARα Signaling

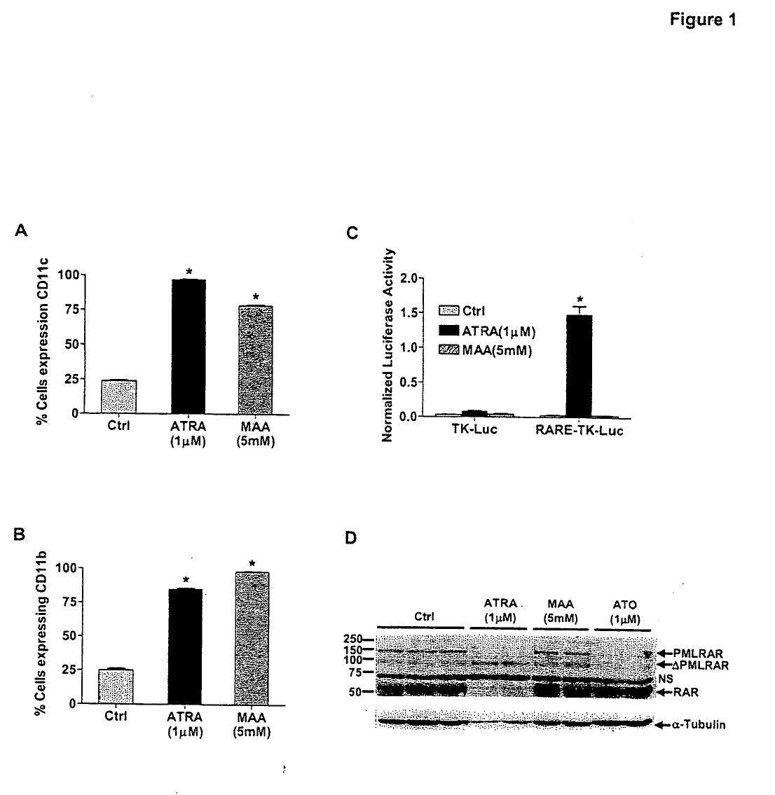

HDAC inhibitors, such as BA and its derivatives, have been used to relieve the differentiation block in APL cells although the precise mechanism(s) by which these agents manifest their activity is unclear (Kramer et al, Trends Endocrinol. Metab. 12:294-300 (2001)). It has been established previously that MAA, a compound chemically related to BA, can function as inhibitors of class I HDACs (Jansen et al, Proc. Natl. Acad. Sci. USA 101:7199-7204 (2004)). However, these compounds have additional pharmacological activities that may contribute to their efficacy in cellular models of APL. Of particular interest in this regard was the observation that MAA could function as both an inhibitor of HDACs and as an activator of MAPK. Together these activities enabled this compound to increase the transcriptional efficacy of nuclear hormone receptors in vitro and in vivo (Jansen et al, Proc. Natl. Acad. Sci. USA 101:7199-7204 (2004)). The role of the HDACI activity of MAA, and related compounds, in APL differentiation was probed with a view to determining whether or not other activities of these compounds contribute to their efficacy in leukemic cells. MAA was chosen for these studies as it has a long half-life in vivo (Shih et al, Arch. Environ. Health 56:20 (2001)). As shown in FIGS. 1A and 1B, MAA is able to increase the percentage of NB4 cells, an APL cell line, expressing the myeloid differentiation markers CD11c and CD11b to a level comparable to ATRA, a well-characterized differentiating agent. Given these properties, attention next turned to defining the biochemical process targeted by MAA and related compounds that is required for APL cell differentiation.

In APL, the PML-RARα fusion protein within NB4 cells is thought to function as a dominant negative inhibitor of normal RARα signaling, resulting in the silencing of retinoic acid response element (RARE)-dependent gene transcription (Melnick and Licht, Blood 93:3167-3215 (1999). The ability of pharmacological doses of ATRA to promote NB4 cell differentiation is most likely the result of its ability to relieve the transcriptional repression associated with PML-RARα (Melnick and Licht, Blood 93:3167-3215 (1999). Not surprisingly therefore, ATRA at 1 μM can activate transcription of an RARE-containing reporter gene (RARE-TK-Luc) in NB4 cells (FIG. 1C). However, although MAA at 5 mM alone can induce expression of CD11c and CD11b (FIGS. 1A and 1B), it does not activate the RARE-TK-Luc reporter (FIG. 1C).

Both ATRA and ATO treatments can induce PML-RARα degradation whereas ATRA alone has the additional property that it can reduce the expression of the RARα protein (Chen et al, Blood 89:3345-3353 (1997), Raelson et al, Blood 88:2826-2832 (1996)). Degradation of PML-RARα by ATRA is accompanied by the accumulation of a 90-KDa cleavage product (ΔPML-RARα) whereas ATO induces complete degradation of PML-RARα (Chen et al, Blood 89:3345-3353 (1997), Zhu et al, Proc. Nat. Acad. Sci. USA 96:14807-14812 (1999)). To verify whether RARα- or PML-RARα-related pathways are involved in MAA-induced differentiation, we analyzed the expression of PML-RARα and RARα proteins in NB4 cells was analyzed by Western blot analysis. As shown in FIG. 1D, while ATRA and ATO induced the degradation of the PML-RARα fusion protein in NB4 cells, MAA had no effect on the expression level of this protein. The ATRA-induced degradation of RARα and accumulation of ΔPML-RARα were also not observed in MAA-treated samples. These results suggest that MAA-induced differentiation does not require the degradation of RARα or PML-RARα.

MAA Induces Phosphorylation of C/EBPβ at Thr-235, Which Requires ERK Activity in NB4 Cells

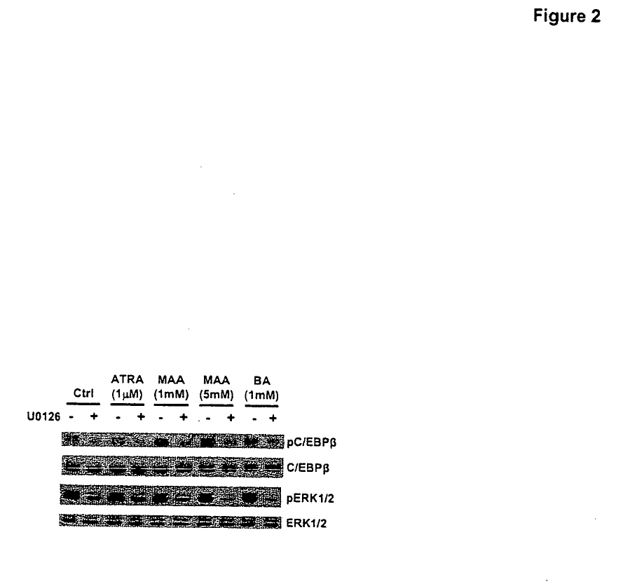

To identify the pathway(s) activated by MAA that are involved in NB4 cell differentiation, a determination was made as to whether MAA targets transcription factors, other than RARα and PML-RARα, that have previously been implicated in promyelocyte differentiation. One such factor, C/EBPβ plays an important role during differentiation of a number of cell types including myeloid cells (Scott et al, Blood 80:1725-1735 (1992)). During myeloid cell development, the expression of C/EBPβ increases and positively regulates the tissue specific activation of the CD11c promoter (Lopez-Rodriguez et al, J. Biol. Chem. 272:29120 (1997); Scott et al, Blood 80:1725 (1992)). Although C/EBPβ expression has been shown to be required for ATRA-induced differentiation in APL cells (Duprez et al, Embo J. 22:5806-5816 (2003)), the importance of C/EBPβ phosphorylation in promyelocyte differentiation has not been investigated. It has been shown that C/EBPβ is phosphorylated at Thr-235 by ERK1/2, resulting in an enhancement of its transcriptional activity (Nakajima et al, Proc. Natl. Acad. Sci. USA 90:2207-2211 (1993), Piwien-Pilipuk et al, J. Biol. Chem. 277:44557-44565 (2002)). It has been previously determined that MAA can activate ERK1/2 (Jansen et al, Proc. Natl. Acad. Sci. USA 101:7199-7204 (2004)), and thus an examination was made here as to whether phosphorylation of C/EBPβ at Thr-235 was influenced by treatment with this compound in NB4 cells. As shown in FIG. 2, a dose-dependent increase in the level of phosphorylated C/EBPβ was observed in MAA-treated cells. Similarly, BA, a well-characterized HDACI that also activates ERK (Yang et al, J. Biol. Chem. 276:25742-25752 (2001)), increases phosphorylation of C/EBPβ at Thr-235. Treatment of NB4 cells with the MEK1/2 inhibitor U0126 blocks MAA- or BA-induced phosphorylation of C/EBPβ at Thr-235 without changing the absolute level of the protein (FIG. 2). These results suggest that HDACI-mediated APL cell differentiation may require an ERK-dependent regulation of C/EBPβ phosphorylation. However, since ERK is constitutively active in NB4 cells and is not further activated by either MAA or BA, it appears that their effects on ERK-dependent C/EBPβ phosphorylation occur in an indirect manner.

MAA Exerts a Dose-dependent Dual Effect on NB4 Cell Differentiation and Apoptosis

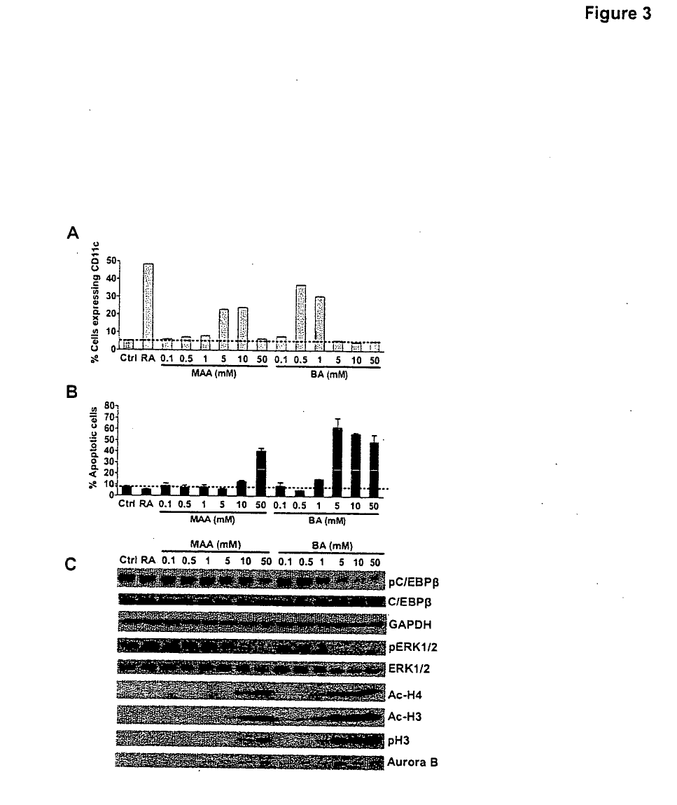

Next a closer examination as made of the dose-dependent effect of MAA on C/EBPβ phosphorylation and NB4 cell differentiation. NB4 cells were treated with 0.1 to 50 mM of MAA and the percentage of cells expressing the differentiation marker CD11c was examined using flow cytometry. As shown in FIG. 3A, the maximum percentage of cells expressing CD11c was observed at between 5 mM and 10 mM MAA with a considerable diminution of activity being observed at higher concentrations. Similarly, NB4 cells treated with 5 mM (or higher) BA also failed to differentiate although differentiation was observed in cells treated with 0.5 mM and 1 mM of the compound (FIG. 3A). Interestingly, maximal phosphorylation of C/EBPβ at Thr-235 was observed in cells treated with lower concentrations of either MAA or BA (FIG. 3C). At higher concentrations of these HDACIs, the level of phospho-C/EBPβ in cells was markedly reduced (FIG. 3C). No significant change in the level of total C/EBPβ was observed at all the concentrations of HDACIs examined in this study (FIG. 3C).

Since HDACIs have also been shown to induce apoptosis of many cancer cells including leukemia cells, the percentage of cells that bind to the apoptosis marker Annexin V in NB4 cells was examined. As shown in FIG. 3B, as the expression of CD11c decreased at higher concentrations of HDACIs (FIG. 3A), the percentage of cells undergoing apoptosis increased. Similarly, suberoylanilide hydroxamic acid (SAHA), an HDACI chemically unrelated to MAA or BA, also effectively induced apoptosis in NB4 cells at higher concentrations and facilitated differentiation when lower concentrations were added (data not shown). Thus, differentiation and apoptosis can be pharmacologically uncoupled in APL cells, suggesting the targets of HDACIs in these two processes are unlikely to be same.

At Higher Concentrations HDACIs Decrease ERK Activity, Increase Global Histone Acetylation and Phosphorylation, and Increase Aurora Kinase Expression in NB4 Cells

These studies implicate the MAPK signaling cascade in HDACI-mediated increases in C/EBPβ phosphorylation and APL differentiation. Thus, the role of the MAPK pathway in the apoptotic events observed in cells treated with the highest concentrations of MAA and BA was examined. As shown in FIG. 3C, NB4 has a high steady-state level of phosphorylated ERK that remains detectable even after 48 hours of serum starvation (data not shown). Lower concentrations of MAA or BA did not change the constitutive levels of phosphorylated ERK. However, when MAA or BA was used at higher concentrations (those at which no differentiating effects can be observed) the levels of phosphorylated ERK, but not total ERK protein were dramatically decreased (FIG. 3C).

To examine the extent to which the HDAC inhibitor activity of MAA or BA is involved in the differentiation or apoptosis of NB4 cells, acetylation of histones H3 and H4 was analyzed by Western blot analysis. As shown in FIG. 3C, at concentrations of MAA optimal for differentiation no significant increase in H3 or H4 acetylation was observed. However, robust acetylation of these proteins was observed at the concentrations of MAA and BA required for apoptosis in these cells. These results suggest that substantial histone acetylation, coupled with global derepression of transcription, is required for HDACI-mediated apoptosis and that a more defined set of targets may be sufficient to induce differentiation. It is possible therefore that at low concentrations, biochemical activities of HDACIs other than regulation of histone acetylation may play a role in APL cell differentiation.

Phosphorylation of H3 at Ser-10 is associated with transcription and can be induced by various stimuli, including epidermal growth factor (EGF) and apoptosis-inducing agents (Clayton and Mahadevan, FEBS Lett. 546:5-58 (2003), Wang and Lippard, J. Biol. Chem. 279:206922-206225 (2004), Waring et al, J. Biol. Chem. 272:17929-17936 (1997)). Therefore, an examination was made as to whether H3 phosphorylation could be associated with either NB4 cell differentiation or apoptosis. As shown in FIG. 3C, treatment of NB4 cells with higher concentrations of MAA or BA dramatically increased H3 phosphorylation whereas lower concentrations of MAA or BA were without effect. These results indicate that like acetylation, phosphorylation of H3 is linked to the apoptosis but not to the differentiation-promoting effects of MAA and BA in NB4 cells.

Phosphorylation of H3 at Ser-10 has been shown to be mediated through ERK or p38-mediated pathways during stimuli-induced transcription and through Aurora kinases during mitotsis (Nowak and Corces, Trends Genet 20:214-220 (2004), Prigent and Dimitrov, J. Cell. Sci. 116:3677-3685 (2003)). However, ERK activity is downregulated at the concentrations at which H3 phosphorylation was observed (FIG. 3C), suggesting that ERK may not be the kinase involved. p38 was also not required for H3 phosphorylation since its activity was not affected by HDACIs and treatment of NB4 cells with a p38 inhibitor, SB203580, has little effect on apoptosis or H3 phosphorylation at Ser-10 (data not shown). Interestingly, examination of Aurora B revealed that its expression was absent in untreated NB4 cells but greatly induced with higher concentrations of HDACIs (FIG. 3C), suggesting that this kinase may be involved in phosphorylating H3 in NB4 cells.

Downregulation of ERK Activity Inhibits Differentiation and Induces Apoptosis of NB4 Cells

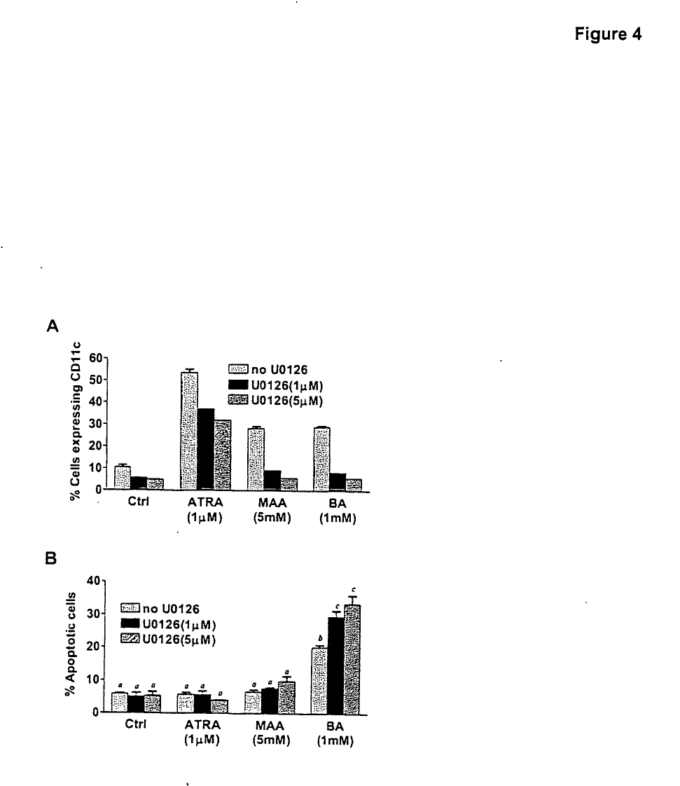

Since higher concentrations of HDACIs repress differentiation and induce apoptosis, an activity that correlates with downregulation of ERK activity in NB4 cells (FIG. 3), the effect of MEK/ERK inhibition on these processes was next assessed directly. For these studies, NB4 cells were treated with ATRA, MAA, or BA with or without MEK inhibitor U0126 and the percentage of cells undergoing differentiation or apoptosis were analyzed. The data presented in FIG. 4 showed that treatment of NB4 cells with ATRA (1 μM), MAA (5 mM) or BA (1 mM) can induce NB4 cell differentiation whereas BA at 1 mM also effectively induces apoptosis. Inhibition of MEK/ERK activity by U0126 dramatically reduced both MAA and BA-dependent differentiation as assessed by CD11c expression. Inhibition of ERK had a less dramatic effect on ATRA-induced differentiation in these cells. Importantly, treatment of NB4 cells with U0126 also significantly increased the number of BA-treated cells undergoing apoptosis (FIG. 4B). These results provide evidence that ERK activity is required for differentiation of NB4 cells and can protect cells from apoptosis.

MAA Exhibits a Biphasic Dose-dependent Effect on Apoptosis and Differentiation in Non-APL Myeloid Cells

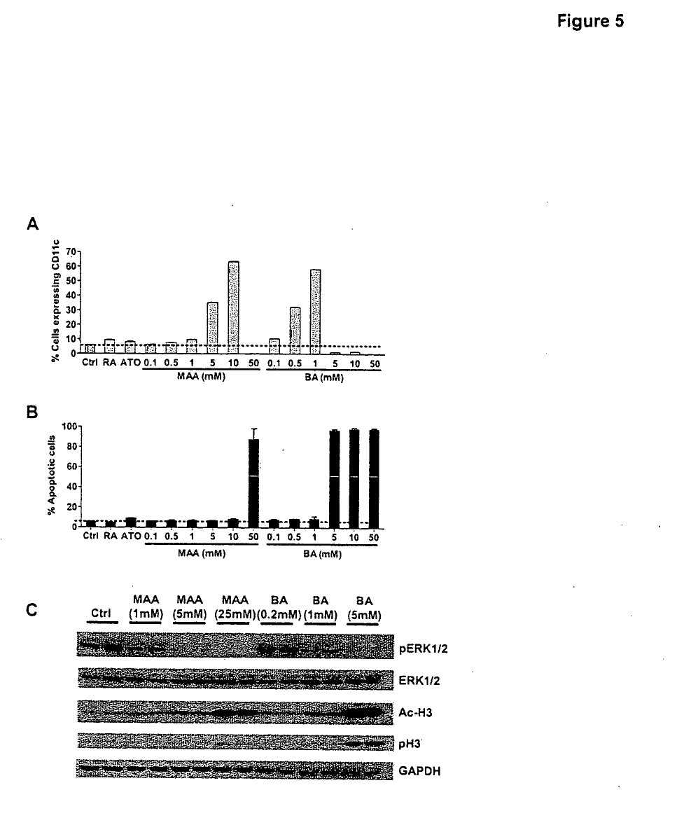

To investigate whether MAA can induce differentiation or apoptosis in cells other than NB4, its effect in the non-APL myeloid cell line U-937 was tested. The U-937 cell line was derived from the pleural effusion of a patient with histiocytic lymphoma (Sundstrom and Nissson, Int. J. Cancer 17:565-577 (1976)) and has been used as a myeloid differentiation model. The data in FIGS. 5A and 5B show that MAA and BA exhibit a similar dose-dependent dual effect on U937 cells, increasing expression of the differentiation marker CD11c at lower concentrations and inducing apoptosis at higher concentrations. Higher concentrations of MAA or BA also downregulate the activity of ERK1/2 without affecting the total ERK1/2 protein levels (FIG. 5C). This is also correlated with an increase in H3 acetylation and phosphorylation (FIG. 5C). These results suggest that HDACIs regulate similar signaling events in cells other than NB4 cells.

MAA Induces Differentiation in Both PML-RARα-negative Leukemia Cells and ATRA-resistant APL Cells

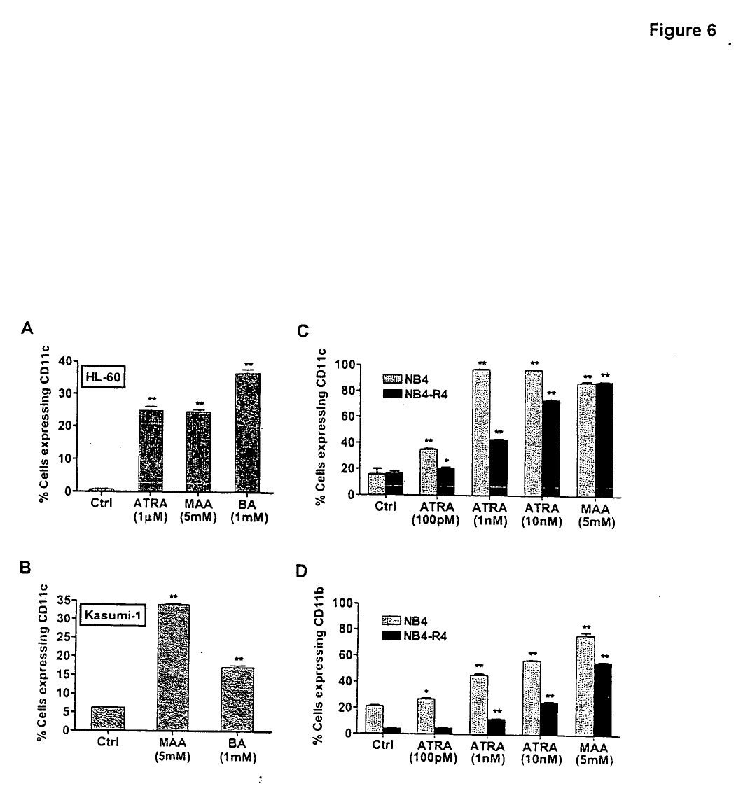

Since MAA does not directly target PML-RARα in NB4 cells (FIG. 1) and it can induce differentiation in non-APL cells (FIG. 5), a further investigation was made as to whether MAA can induce differentiation in different myeloid leukemia cells or in APL cells that have become resistant to ATRA treatment. HL-60 cells are an APL cell line that does not possess the t(15;17) translocation although they can be induced to mature along the myeloid lineage by ATRA (Breitman et al, Proc. Natl. Acad. Sci. USA 77:2936-2940 (1980)). In these cells, both MAA and BA were found to induce differentiation as evidenced by the expression of the CD11c (FIG. 6A). Kasumi-1 cells are a model of AML with a t(8;21) chromosomal translocation, which fuses the AML-1 (Acute Myeloid Leukemia 1) DNA-binding transcription factor to the ETO (eight-twenty-one) corepressor that associates with HDAC complexes (Wang et al, Proc. Natl. Acad. Sci. USA 95:10860-10865 (1998)). It is believed that the recruitment of a corepressor/HDAC to AML1/ETO can block the transactivation of AML-1-dependent target genes. Therefore, a test was made to determine if MAA or BA can reverse this transcriptional repression and induce differentiation of these cells. As shown in FIG. 6B, MAA and BA both increased the percentage of cells expressing CD11c in this cell line. These results support the hypothesis that HDACIs like MAA can be used in the treatment of leukemias other than those that are PML-RARα-positive.

Although most APL patients respond initially to ATRA treatment, some will eventually develop resistance to this agent. Resistance in APL cells has been correlated with a loss of the expression of the PML-RARα fusion protein (Fanelli et al, Blood 93:1477-1481 (1999)) or mutations within this protein which display reduced ATRA binding (Shao et al, Blood 89:4282-4289 (1997)). Thus, the ATRA-resistant NB4-R4 cell line was used to test whether MAA could be an effective therapy in patients who have progressed on ATRA. NB4-R4 cells were derived by continuous culturing of NB4 cells in ATRA-containing media (Rosenauer et al, Blood 88:2671 (1996)). The PML-RARα protein in NB4-R4 cells contains a point mutation in the ligand binding domain that reduces its ability to bind to retinoic acid(s) (Shao et al, Blood 89:4282-4289 (1997)). However, the mutant PML-RARα can still bind to retinoic acid response elements and thus functions as a dominant negative inhibitor of transcription which is not relieved by retinoic acid (Rosenauer et al, Blood 88:2671-2682 (1996), Shao et al, Blood 89:4282-4289 (1997)). Using the differentiation markers CD11c (FIG. 6C) and CD11b (FIG. 6D), it was demonstrated that NB4-R4 cells are indeed less sensitive to ATRA treatment than the NB4 parental line. However, MAA at 5 mM can increase the percentage of cells expressing CD11c (FIG. 6C) and CD11b (FIG. 6D) to a level similar to that observed in NB4 cells . Thus, low concentrations of MAA can circumvent the inhibitory activity of both wild-type and mutant PML-RARα, allowing these APL cells to differentiate.

MAA Potentiates ATRA and ATO-induced Differentiation or Apoptosis in NB4 Cells

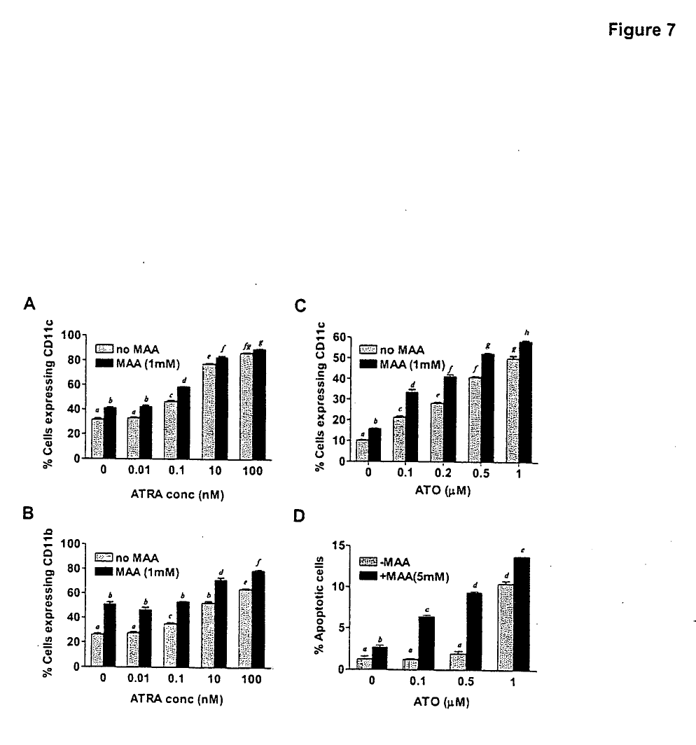

Although treatment of APL patients with ATRA results in high rates of complete clinical remission, the use of ATRA causes serious systemic toxicity (Tallman et al, Blood 95:90-95 (2000)). Thus, drugs that enhance the activity of ATRA could be useful in the treatment of these patients by reducing the doses of retinoid that need to be administered. As shown in FIG. 7A, treatment of NB4 cells with 1 nM MAA can increase the percentage of cells expressing CD11c from 32% to 41%. The effect is of the same order of magnitude as that observed following treatment with 0.1 nM of ATRA. However, when cells are simultaneously treated with 1 nM MAA and 0.1 nM ATRA, the percentage of cells expressing CD11c was increased to 60%. Similar potentiation of ATRA activity by MAA was also observed when the expression of CD11b was assessed (FIG. 7B). Whereas MAA (1 mM) or ATRA (10 nM) alone increased the percentage of cells expressing CD11b from 26% to 51%, combination of both reagents increased it to 71%.

An examination was also made of the possible utility of combining MAA and ATO for treatment of APL. ATO can trigger apoptosis of NB4 cells at high concentrations (0.5 to 2 μM) and induce differentiation at low concentration (0.1 to 0.5 μM) (Chen et al, Blood 89:3345-3353 (1997). As shown in FIG. 7C, MAA at 1 mM significantly increases the percentage of cells expressing CD11c in NB4 cells induced by all of the concentrations of ATO tested (0.1 to 1 μM). MAA at 5 mM or ATO (0.1 μM, 0.5 μM) does not induce significant amount of apoptosis, however, the combination of MAA and ATO dramatically increases the percentage of cells undergoing apoptosis (FIG. 7D). These results suggest additional utilities of HDACIs in that they can be used to (a) increase the efficacy of established drugs and (b) ameliorate the side effect profile of ATO-based therapies by reducing the doses needed to effect a response.

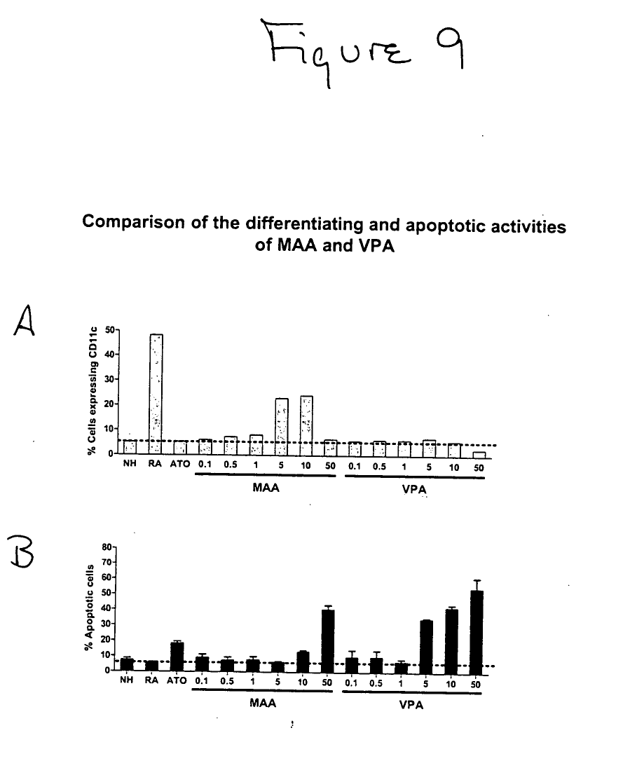

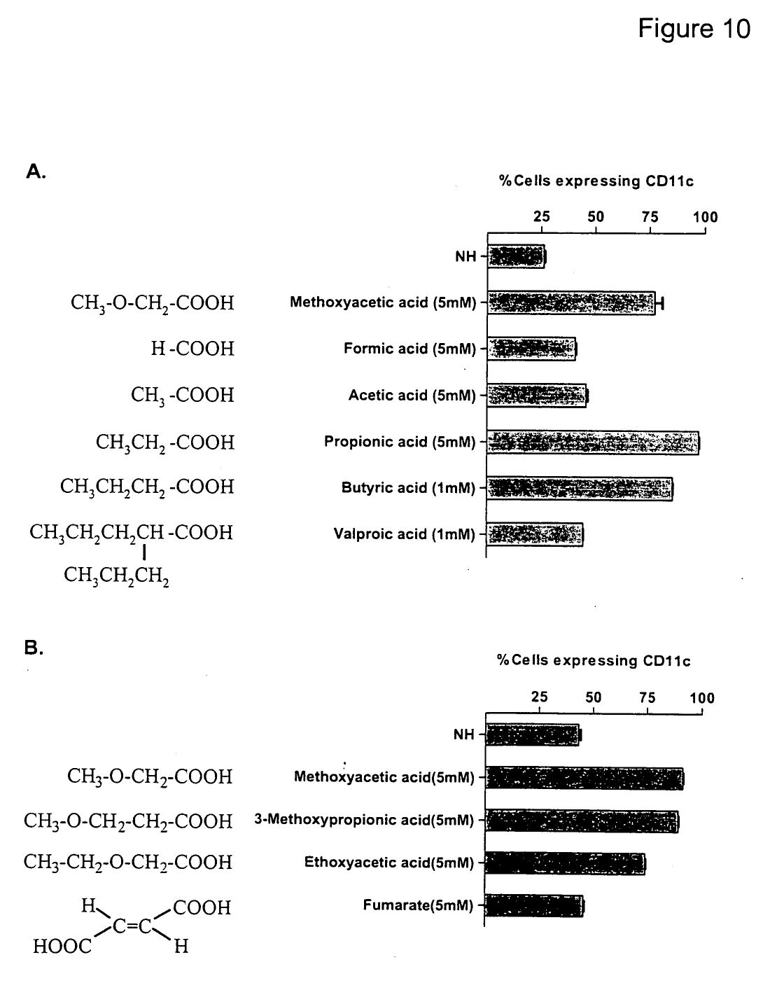

Differentiating activities of several short chain fatty acids with structures imilar to MAA were examined. Like MAA, propionic acid, butyric acid, 3-methoxypropionic acid and ethoxyacetic acid are potent inducers of NB4 differentiation. The dual effect of MAA and butyric acid on differentiation acid apoptosis in NB4 cells has not yet been demonstrated with other short chain fatty acids shown in FIG. 10. As shown in FIG. 9, although VPA potently induces apoptosis as concentrations increase, it did not significantly increase the percentage of NB4 cells expressing CD11c. Thus if differentiation therapy is the preferred activity in treating, for example, APL, use of an alternative short chain fatty acid may be selected.

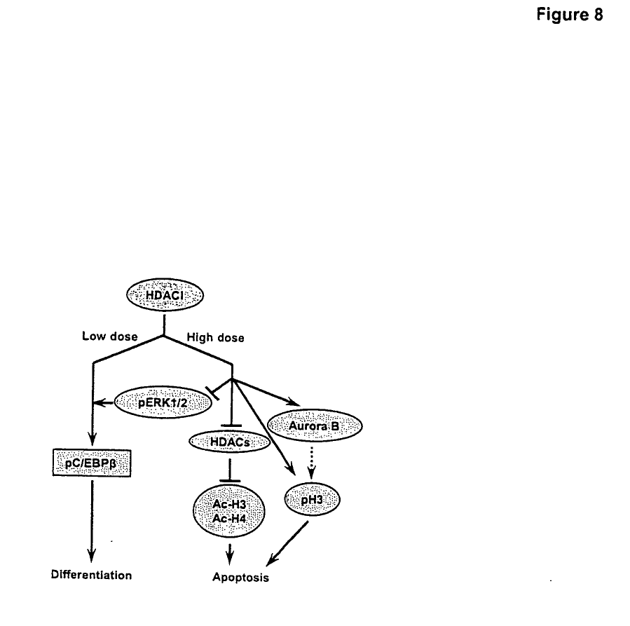

Summarizing, in this study, it has been shown that HDACIs induce differentiation and apoptosis in myeloid leukemic cell lines by distinct mechanisms (FIG. 8). At concentrations where these agents are unable to effect a measurable effect on histone acetylation, they can induce differentiation. This event mirrors a robust increase in the phosphorylation of C/EBPβ a transcription factor implicated previously in myeloid differentiation. At higher concentrations, the HDACIs tested induce apoptosis and increase histone acetylation/phosphorylation but importantly they also downregulate ERK activity (phosphorylation) in these cells. Importantly, ERK is constitutively active in NB4 cells, and it was possible to demonstrate that it was required for both C/EBPβ phosphorylation and differentiation, and that it also protects cells from undergoing apoptosis. Inhibition of ERK activity did not abrogate the proapoptotic activity of high concentrations of HDACIs and had very minimal effects on histone acetylation or phosphorylation (data not shown). These data strongly suggest that ERK is required for HDACI-mediated differentiation of APL cells and that these compounds manifest their proapoptotic activities in an ERK-independent manner (FIG. 8).

Induction of apoptosis by high concentrations of HDACIs correlates very well with the increased acetylation of histones H3 and H4, suggesting that it is the ability of these agents to facilitate a global derepression of gene transcription that may be responsible for the apoptotic activity of these compounds in NB4 cells. The question arises, however, as to the mechanism(s) underlying the activity of low concentrations of HDACIs in differentiation. It is possible that these compounds do indeed facilitate the acetylation of histones to some degree and permit the upregulation of a small sub-set of genes that facilitate differentiation. It is also possible that they effect the acetylation of a non-histone factor that ultimately impacts the phosphorylation of C/EBPβ. An attempt has been made, without success, to show that C/EBPβ acetylation is increased by HDACIs in cells. Finally, it is possible that low dose HDACIs have effects on cell signaling pathways in a manner that is independent of their ability to inhibit HDACs. Indeed, it was previously shown in several cell lines that MAA and BA can activate ERK and enhance the transcriptional activity of nuclear receptors (Jansen et al, Proc. Natl. Acad. Sci. USA 101:7199 (2004)). Although ERK is constitutively active in the APL cells, the observation that these agents can activate ERK provides a precedence for these potential “off-target” effects.

In probing the mechanism by which higher concentrations of HDACIs facilitate apoptosis it was observed that they induce a robust of phosphorylation of H3 at Ser-10, an event that is usually associated with transcriptional activation or mitotic chromosome condensation (Peterson and Laniel, Curr. Biol. 14:R546-551 (2004)). However, several cases have been reported that H3 phosphorylation at Ser-10 is implicated in apoptosis. For example, this H3 phosphorylation was observed in thymocytes that were induced to apoptose by using gliotoxin Waring et al, J. Biol. Chem. 272:17929-17936 (1997)). In addition, the pro-apoptotic drug cisplatin can also induce H3 phosphorylation at Ser-10 in HeLa cells Wang and Lippard, J. Biol. Chem. 279:206922-206225 (2004)). Furthermore, ATO was shown to promote H3 phosphorylation at Ser-10 in APL cells (Li et a, J. Biol. Chem. 277:49504-49510 (2002)). Thus, it is possible that this specific histone modification may play an important role in the apoptotic effect of some antileukemic agents. Interestingly, expression of Aurora B, a mitotic H3 kinase, correlated very well with the HDACI-induced H3 phosphorylation and apoptosis in NB4 cells, suggesting that Aurora kinase might be required for these processes. However, it remains to be determined if increased H3 phosphorylation is due to the increased Aurora B expression or they are independent activities of HDACIs.

Examination of ERK activity in NB4 cells revealed that these cells express high basal levels of phosphorylated ERK that is not induced any further with differentiating doses of HDACIs. With apoptotic concentrations of HDACIs, however, a dramatic decrease in the levels of phosphorylated ERK in NB4 cells was observed. It is possible that higher concentrations of HDACIs may induce the expression of a MAPK phosphatase (MKP), which then in turn inactivates ERK (Theodosiou and Ashworth, Genome Biol. 3:Reviews3009 (2002)). However, treatment of NB4 cells with a tyrosine phosphatase inhibitor sodium orthovanadate did not inhibit the apoptosis of NB4 cells in response to HDACI treatment (data not shown). Examination of several MKPs including MKP-1, MKP-3, MKP-4, and PAC-1 also did not show any significant increase in their expression in response to apoptotic doses of HDACIs.

Although HDAC inhibitors have been shown to induce differentiation and apoptosis in a number of leukemia cell lines (Kramer et al, Trends Endocrinol. Metab. 12:294-300 (2001)), some HDAC inhibitors are of limited use due to poor bioavailability in vivo. For example, TSA is a potent HDAC inhibitor and exhibits anti-tumor activity in vitro but is rapidly metabolized and does not exhibit significant activity in vivo (Qiu et al, Br. J. Cancer 80:1252-1258 (1999), Sanderson et al, Drug Metab. Dispos. 32:1132-1138 (2004)). BA and phenylbutyrate are well-tolerated in humans but high drug concentrations in plasma are difficult to maintain due to their short half-life in vivo (BA: t_{1/2}=6 min; Phenylbutyrate: t_{1/2}=1 h) (Daniel et al, Clin. Chim. Acta. 181:255-263 (1989), Dover et al, Blood 84:339-343 (1994), Qiu et al, Br. J. Cancer 80:1252-1258 (1999), Sanderson et al, Drug Metab. Dispos. 32:1132-1138 (2004)). MAA is a metabolite of ethylene glycol monomethyl ether, an industrial solvent shown to be a developmental toxicant (Miller et al, Fundam. Appl. Toxicol. 2:158-160 (1982), Nagano et al, Toxicology 20:335-343 (1981), Scott et al, Teratology 39:363-373 (1989)). The elimination half-life of MAA has been determined to be longer than other HDAC inhibitors (77.1 h in human, 20 h in non-human primates, and 13-18 h in rats) (Aasmoe et al, Xenobiotica 29:417-424 (1999), Scott et al, Teratology 39:363-373 (1989), Shih et al, Arch. Environ. Health 56:20-25 (2001)), suggesting that MAA is a pharmacologically more stable compound. MAA is clearly a less potent HDACI than BA as shown in our study. However, given the observation that HDAC inhibition correlates with apoptosis and not differentiation, MAA may actually be superior if a true differentiation therapy is the goal. Whether the clinical outcome of cancers treated with agents that favor differentiation or apoptosis is different remains to be determined.

It is generally considered that PML-RARα aberrantly recruits an HDAC-corepressor complex to the RARα target gene promoters, causing their silencing and thus generating a differentiation block. Specifically, it is proposed that pharmacological doses of ATRA relieve transcriptional repression by disrupting the activity of the HDAC/corepressor associated with PML-RARα thus allowing the recruitment of coactivators (Lin et al, Nature 391:811-814 (1998)). In support of this hypothesis, it has been shown that HDACIs can synergize with ATRA in the activation of RARα target gene promoter transcription and facilitate differentiation of APL cells (Lin et al, Nature 391:811-814 (1998)). However, HDACIs have also been shown to induce differentiation and apoptosis in non-APL leukemic cells (Drummond et al, Annu. Rev. Pharmacol. Toxicol. 45:495-528 (2005)), suggesting that PML-RARα may not be the only target for HDACI-mediated differentiation of myeloid cells. In this study, it is shown that MAA has a positive impact on both ATRA- and ATO-mediated differentiation and apoptosis in NB4 cells. However, MAA alone also exhibits substantial differentiating activity in a manner that does not involve PML-RARα-mediated transcriptional activity and which occurs at concentrations where no significant increased histone acetylation was observed. These findings attest to the pharmacological complexity of this series of compounds and indicate that PML-RARα is not the primary target of HDACIs in either ATRA sensitive or resistant APL.

In conclusion, these studies have determined that depending on the level of HDAC inhibition, leukemic cells can either differentiate or undergo apoptosis. Furthermore, the observation that quantitative inhibition of HDACs leads to a decrease in the phosphorylation of C/EBPβ, a factor required for differentiation, indicates that the mechanisms underlying HDACI-mediated differentiation and apoptosis are distinct. Indeed the findings clearly demonstrate that HDACIs have activities in cells beyond HDAC inhibition providing a strong rationale to test the therapeutic efficacy of different doses of HDACIs in patients. Given its excellent pharmaceutical properties and its relatively weak HDACI activity, MAA itself may prove to be useful to probe this alternative therapeutic approach.

All documents and other information sources cited above are hereby incorporated in their entirety by reference.

What is claimed is:

1. A method of treating a hematological malignancy comprising administering to a patient in need thereof an amount of at least one short chain fatty acid, or pharmaceutically acceptable salt thereof, that is both a mitogen-activated kinase (MAPK) activator and a histone deacetylase (HDAC) inhibitor sufficient to effect said treatment.

2. The method according to claim 1 wherein said hematological malignancy is a leukemia.

3. The method according to claim 2 wherein said leukemia is acute promyelocytic leukemia (APL).

4. The method according to claim 2 wherein said leukemia is acute myeloid leukemia (AML) or other undifferentiated leukemia.

5. The method according to claim 2 wherein said leukemia is a retinoic acid (ATRA)-resistant APL.

6. The method according to claim 1 wherein said patient is a human.

7. The method according to claim 1 wherein said method further comprises administering at least one of ATRA and arsenic trioxide (ATO).

8. The method according to claim 1 wherein said short chain fatty acid is a C₃-C₁₂ fatty acid.

9. The method according to claim 8 wherein said short chain fatty acid is a C₃-C₁₀ fatty acid.

10. The method according to claim 9 wherein said short chain fatty acid is a C₃-C₈ fatty acid.

11. The method according to claim 1 wherein said short chain fatty acid is a methoxyacetic acid (MAA), butyric acid (BA), valproic acid (VPA), propionic acid, 3-methoxypropionic acid or ethoxyacetic acid, or pharmaceutically acceptable salt thereof.

12. A composition comprising: i) at least one short chain fatty acid, or pharmaceutically acceptable salt thereof, that is both a MAPK activator and a HDAC inhibitor, and ii) at least one of ATRA and ATO.

13. A method of reducing the number of preneoplastic cells in a patient in need thereof comprising administering to said patient an amount of at least one short chain fatty acid, or pharmaceutically acceptable salt thereof, that is both a MAPK activator and a HDAC inhibitor sufficient to effect said reduction.

14. A kit comprising at least one short chain fatty acid, or pharmaceutically acceptable salt thereof, that is both a MAPK activator and a HDAC inhibitor disposed within a container means and at least one of ATRA and ATO disposed within a container means.

15. A method of identifying compounds potentially suitable for use in treating leukemia comprising assaying candidate compounds for their the ability to increase the percentage of AML cell models that express myeloid differentiation markers CD11b and CD11c, wherein compounds that effect said increase are potentially suitable for use in treating leukemia.

16. The method according to claim 15 wherein said assaying is effected using flow cytometry analysis.
